(19) United States
(12) Patent Application Publication
(10) Pub. No.: US 2020/0327990 A1
(43) Pub. Date: Oct. 15, 2020

(54) SYSTEMS, APPARATUSES, AND METHODS FOR ASSESSING, MANAGING, PRESENTING AND INDICATING HEALTH STATUS THROUGH PHYSICAL OBJECTS

(71) Applicant: Blockchain Goose Inc., San Jose, CA (US)

(72) Inventor: Kianoosh MOUSAVI, San Jose, CA (US)

(21) Appl. No.: 16/617,446

(22) PCT Filed: Apr. 9, 2019
(86) PCT No.: PCT/US19/26609

§ 371 Date: Nov. 26, 2019

Related U.S. Application Data
(63) Continuation-in-part of application No. 16/270,326, filed on Feb. 7, 2019.
(60) Provisional application No. 62/655,535, filed on Apr. 10, 2018.

(51) Int. Cl. G16H 50/30; G16H 10/60; G16H 50/20; G16H 40/67; G06F 21/62; G06F 3/0484

(52) U.S. Cl. CPC G16H 50/30; G16H 10/60; G06F 3/0484; G16H 40/67; G06F 21/6245; G16H 50/20

(57) ABSTRACT

Apparatuses and methods for physically holding, presenting, verifying, exchanging and/or displaying the health status of an individual which may include a human interface to physically present and/or display the value of at least one set of health metrics the apparatuses represent. The apparatuses may include a set of logical circuits coupled with a specialized memory to temporary or permanently store the health metrics and/or health data information and determine the display variation of their the wearer's health status on the human interface based on the health metric type and value. The apparatuses may also include a set of connectors to access the sources of the health metrics and update the health index value the apparatuses represent. The apparatuses may also include a power source to power and enable the capabilities mentioned above.

### CROSS-REFERENCE TO RELATED APPLICATIONS

This application claims priority to applicant's co-pending U.S. Provisional Application No. 62/655,535 titled “System and Method for Representing Health Status Through Physical Objects,” filed Apr. 10, 2018, and to U.S. patent application Ser. No. 16/270,326 titled “Systems, Apparatuses, and Methods for Assessing, Managing, Presenting and Indicating the Value of a Set of Digital Assets,” filed Feb. 7, 2019. The entirety of each of the foregoing applications is incorporated herein by reference in its entirety.

### FIELD OF DISCLOSURE

This disclosure relates in general to representing an individual's health status or condition, and in particular to an apparatus for outwardly representing how healthy or physically fit an individual is based on various health metrics.

### BACKGROUND

In general, there are individuals who enjoy the conspicuous display of items with intrinsic value, such as gold or diamonds.

Conspicuous consumption is generally spending of money on and the acquiring of luxury goods and services to publicly display accumulated wealth of the buyer. Often this display is a means of either attaining or maintaining a given social status.

Another less tangible yet equally important marker of social status is an individual's health and physical fitness, particularly with respect to social activities like dating.

While some information on a particular individual's health status may be gained from observation, many chronic or contagious diseases have no outward physical manifestations or outwardly detectable symptoms. For example, one would not know from mere brief observation if a particular individual has a sexually transmitted disease, high blood pressure, high blood sugar, poor V0₂ Max, or other types of chronic conditions, diseases, or physical fitness metrics indicating an individual is in poor health.

Likewise, an individual who is free from chronic conditions or contagious diseases has no way to outwardly manifest their good health.

In view of the foregoing, it would be beneficial to provide an apparatus that verifies and conspicuously displays the health status or condition of the owner or wearer to other parties publicly or privately.

### SUMMARY

Aspects of the present disclosure relate, in one embodiment, to an apparatus that displays a health index score or value that indicates the health status or condition of an individual. The health index score or value may reflect health metrics from various internal or external health data monitoring services, health data providers, medical devices, laboratory test results, or health data applications. The apparatus includes a human interface configured to present an individual's health index value or score. The apparatus also includes a set of logic circuits coupled to the human interface. The apparatus further includes a set of connectors, logically coupled to the set of logic circuits, wherein at least one connector, of the set of connectors, coupled with a health data source to the set of logic circuits; and a power source electrically coupled to the set of logic circuits.

Embodiments further include environmental sensors that affect health such as air pressure, temperature, radiation and air quality. And embodiments further include connecting and exchanging information between apparatuses that are proximate to each other.

Additional advantages and novel features of these aspects will be set forth in part in the description that follows, and in part will become more apparent to those skilled in the art upon examination of the following or upon learning by practice of the disclosure.

### BRIEF DESCRIPTION OF THE DRAWINGS

Aspects of the present disclosure are illustrated by way of example, and not by way of limitation, in the figures of the accompanying drawings and in which like reference numerals refer to similar elements and in which:

FIG. 1 shows a schematic illustration of the apparatus, in accordance with aspects of the present disclosure;

FIG. 2 shows a more detailed illustration of a display in the apparatus, in accordance with aspects of the present disclosure;

FIG. 3 shows a user's interactions with the apparatus and a simplified process flow schematic with an apparatus in accordance with aspects of the present disclosure;

FIG. 4 shows the simplified process flow of FIG. 3 in which a new health metric address, which is a unique identifier of health data endpoints is added to an apparatus and verified, in accordance with aspects of the present disclosure;

FIG. 5 shows a process for generating a new health metric address from a public key, in accordance with aspects of the present disclosure;

FIG. 6 shows a simplified logical diagram of an apparatus, in accordance with aspects of the present disclosure;

FIG. 7 shows a one-way hash function that may be used with health metric data, in accordance with aspects of the present disclosure;

FIG. 8 shows an example of a health metric private key and public address, in accordance with the aspects of the present disclosure;

FIG. 9 shows some specialized Read Only Memory (ROM) or Programmable Read Only Memory (PROM) or One Time Programmable Read Only Memory (OTP) to hold the private key alongside a specialized microcontroller to calculate the public address and a driver to calculate the display value and a Random Access Memory (RAM) to store the values and corresponding dates, for use in accordance with aspects of the present disclosure;

FIG. 10 shows various features of a computer system for use in conjunction with aspects of the present disclosure; and

FIG. 11 shows an example computer system for use in conjunction with aspects of the present disclosure.

FIG. 12 shows how an apparatus interacts with one or more apparatuses to privately exchange health status information between individuals.

### DETAILED DESCRIPTION

Aspects of the present disclosure will now be described in detail with reference to a few preferred embodiments thereof as illustrated in the accompanying drawings. In the following description, numerous specific details are set forth in order to provide a thorough understanding thereof. It will be apparent, however, to one skilled in the art, that aspects of the present disclosure may be practiced without some or all of these specific details. In other instances, well known process steps and/or structures have not been described in detail in order to not unnecessarily obscure description thereof.

In general, it is difficult for a person to manifest his or her authentic health condition or health status of an individual because generally a health conditions or status lack an outward physical appearance.

It is also difficult to uniquely imbue a physical object with authentic health metrics due to the abstract and private nature of those health metrics and the need for those health metrics to be authenticated at a digital device.

In an advantageous manner, a wearable or portable apparatus may be configured, such that a health index value or score derived from health metrics, becomes immutably stored within the apparatus, or accessible by the apparatus, or may be conspicuously presented to, for example, another party, wherein the other party may learn of the wearer's health status by the presented health index value or score.

In one embodiment, an individual's health status or condition may be conveyed through an outward visual or aural representation on the apparatus, wherein the representation may be an indication of a health condition (or absence thereof), the type of condition, the severity of the condition, the contagiousness of the condition, whether the condition is increasing or decreasing and other characteristics of an individual's health condition represented by or held on the apparatus.

In a similar embodiment, the pairing of two or more apparatuses may show compatibility or lack thereof between two individuals by each individual's health index score or value derived from DNA analysis, DNA genealogy, or gene test results or a blood-type reading. For example, such a pairing could reveal that neither individual carries a recessive DNA trait, or that both individuals share a certain genealogy such as Native American, or Ashkenazi.

In another embodiment, the apparatus stores an individual's health status or condition in the form of a health index value or score. Such information may be comprised of data or metrics from health service providers, health applications, or from medical devices, and visually or aurally represents the an individual's current health index value or score through, for example, colors, signs, symbols, brightness or intensity of glow, text, sounds, vibrations or other haptic components, or by any other perceptible feature. The represented health index value or score may be periodically updated by the apparatus after a specified duration.

In yet another embodiment, the apparatus may interact with one or more apparatuses for the purpose of unilaterally or reciprocally exchanging health data or health index scores between the wearers or owners of each apparatus. Health data may be exchanged between apparatuses via a cellular network or Wi-Fi connection to the Internet, or locally via Bluetooth, NFC, ultrasound or audio signal using DTMF or FSK or sound as a carrier wave, or any local connection based on the physical proximity of the apparatuses. The exchange of health information between apparatuses may be encrypted to ensure the privacy of the health information being exchanged. The health data or health index scores of the owners of the connected apparatuses may each be visually or orally represented on the other recipient apparatus.

In a pairing embodiment, an apparatus would normally display an indication of the individual's information. For example, an individual with Native American background may choose to wear an apparatus indicating his authentic Native American heritage. When his apparatus comes in proximity of another individual of that genealogy, the apparatus would make a second indication, such as making a sound or haptic vibration, or displaying a symbol or color, and so could the apparatus of the matching individual nearby.

In yet another embodiment, apparatuses linked via an Internet connection may exchange health data or health index scores on a regular periodic schedule or continuously. Periodic sharing of an individual's health data may be displayed on the recipient's apparatus aurally or visually, with the display showing the health data or health index score as it was on the disclosing apparatus on the date and at the time it was last shared with the recipient device. Continuous sharing of health data or health index scores between apparatus owners may result in displaying the health data or the health index score associated with the disclosing individual's apparatus in real time on the recipient's apparatus.

In yet another embodiment, an individual may configure the apparatus to exchange or share health data or health index scores only with specific recipient apparatuses exclusively. An example of this will be between partners in a monogamous relationship. In this care, an attempt to pair the device with any other will notify the other partner's device of the activity.

In yet another embodiment, the apparatus may be connected through a network, such as the Internet or an intranet, to a health service provider, health data application, or medical device, enabling an individual's health index value or score to be visually or aurally represented in real time. In such embodiment, the apparatus may be used to track any changes in an individual's overall health, physical fitness, or specific medical conditions and visually display the result.

In an alternative embodiment, the apparatus can occasionally be connected to a networked device such as a computer or a mobile phone, which in turn would connect to a source of authentic health-related information to be updated on the apparatus at that time. In the course of such a connection, the information source could authenticate the device, and could require user authentication and only update the information stored on the apparatus following that authentication. Such authentication could transpire by means of the apparatus or the user inputting a private key or user authentication credentials such as a password or a biometric such as a fingerprint or voice signature, and the information source could comprise a public address or even just a website requiring user access credentials to log in.

In another embodiment, the apparatus may be connected through the Internet to a digital health monitoring or testing service or device, enabling the value of the test results to be represented in real time. In such embodiment, the apparatus may also be used to monitor any changes occurring the medical device or value fluctuation of the test result values through visual, aural, or haptic representation.

In another embodiment, the apparatus may be connected to a DNA or genetic testing service account associated with the appratus's wearer or user, enabling identification of a particular genes an individual may carry or other DNA characteristics that may be visually, aurally, or haptically represented by the apparatus.

In another embodiment, an individual's health index value or score can be influenced by health metrics indicating an individual's medical condition, the contagiousness of the condition, the condition's severity, whether the condition is increasing or decreasing, and similar metrics.

In another embodiment, the health index value or score can be influenced by health metrics indicating an individual's level of physical fitness, such as V0₂ max, blood pressure, resting heart rate, muscular strength, and similar indicators of physical fitness.

In another embodiment, the apparatus may further comprise environmental sensors, such as light, air quality, presence of allergens or pollutants, altitude or air pressure, radiation, temperature or humidity. The apparatus may further input that sensor data into calculations including variable data from the health status or information to make an indication to the bearer, or outwardly to others, demonstrating warnings. The warnings could include that the bearer should seek shelter, or avoid contact with others, or descend in elevation.

In another embodiment, the device may keep track of the habitual environment of the wearer, and may include this environmental data in the health index score or value, e.g., weather the wearer lives in a polluted area, which would have adverse effect of on their health or otherwise.

In another embodiment, an externally inaccessible private key is permanently and securely embedded into the apparatus. The inaccessibility of the private key may ensure that any health data transferred to a public address associated with that private key will remain permanently locked in the apparatus and will not be alterable. In such embodiment, the health index value or score displayed on the apparatus can only be adjusted through the inbound transfer of health metric data to the public address of the device, but the historic values will not be alterable. For example, once historic health data values transferred to the apparatus from a source, such as a health application, medical device, or health services provider, are written into a blockchain. Any alteration to an existing health data values stored in the blockchain from the apparatus may require the use of a private key. A unique private key may be generated, for example, from a random seed number associated with the apparatus owner's voice signature. Thus, including a hidden or inaccessible private key in the apparatus may prevent anyone but the apparatus owner from altering, manipulating, or creating false health data values on the apparatus. An example of immutable chain of historical health data may include an owner is diagnosed with cancer and the data is written into the device in a chain, such as a blockchain. Later the owner may have treatment and the cancer is gone to remission or cured. Even though the new information of remission can be added to the history on the device, the original diagnosis cannot be erased or altered without invalidating the complete chain. In this embodiment, the health history of the owner is immutably and securely embedded in the device.

In another embodiment, an accessible private key allows the owner to update the health data history on the chain with new information and revalidate the chain. An example is when the owner once cleared of a disease, such as, a STD or UTI. The owner may be able to remove the diagnosis from the health data history chain and revalidate the chain using the private key.

In yet another embodiment, the apparatus can be different types of jewelry or wearable, such as a bracelet, necklace, watch, ring, pendant, key chain, arm band, etc.

In summary, much like displaying one's social status through the display of expensive jewelry or watches, one's social status may be displayed through an apparatus that outwardly manifests to others is the wearer's authentic health status.

In another embodiment, a personal password or personal identification code may be used to secure access to the health data on the apparatus or update health data on the apparatus, or exchange health data with another apparatus.

In yet another embodiment, biometric sensors like fingerprint identification sensors, retina recognition, facial recognition, or voice authentication, may be used to secure access, and a prerequisite to updating, or exchange health data on the device. These authentication factors may also be used in generating a unique private key for the apparatus that protects the owner's private health data and personal information located thereon. For example, a random seed number may be generated from the owner's voice signature that may be used to generate the unique private key.

In yet another embodiment, the apparatus can hold other immutable values besides health metrics, social media status, or GPA for students.

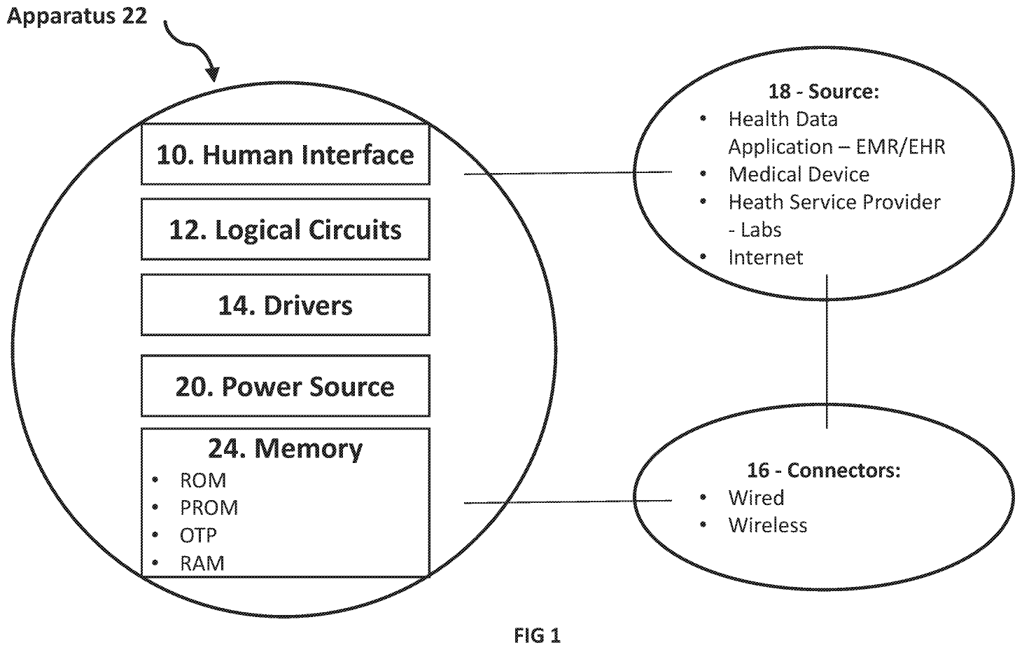

Referring to FIG. 1, a simplified representative schematic of apparatus 22 that allows the health status of the individual, such as a health index value or score the private key of which is stored therein, may be conspicuously presented, in accordance with aspects of the present disclosure. The apparatus may connect via a network, for example, to an external health data application, health service provider, or medical device, or internal device or monitoring system, which may serve as the source of health metrics used to calculate an individual's health index value or score, and the health index value or score of the wearer is visually represented on the apparatus. A Specialized Logic Circuit 12 is coupled with a Memory 24 such as Read Only Memory (ROM) or Programmable Read Only Memory (PROM) or One Time Programmable Read Only Memory (OTP) to hold the private key may operate within or alongside a specialized microcontroller to calculate the public address and a driver to calculate the display health index value or score and a Random Access Memory (RAM) to store the values and corresponding dates.

In general, the apparatus may be comprised of a human interface 10, a set of logic circuits 12 electrically coupled to human interface 10, a set of drivers 14 configured to couple the set of logic circuits 12 to a medical device, medical laboratory, or health data service provider 18, and power source 20 electrically coupled to the set of logic circuits 12.

In another embodiment, the apparatus may also be comprised of environmental sensors that may provide biometric feedback for the purpose of authenticating an apparatus user. For example, the apparatus may contain a fingerprint scanner, voice authentication technology, or facial recognition technology that may be used to secure access to health data on the apparatus.

In one embodiment, human interface 10 comprises a display. In general, “display” means any electronic component that transmits a visually or otherwise perceptible (e.g., audible or tactile) indication corresponding to information, including without limitation one or more of a set of light-emitting diodes (LED), organic light-emitting diodes (OLED), liquid crystal display (LCD) screens, electronic ink or electronic paper displays, a speaker, or other visual or other indicators.

In another embodiment, human interface 10 comprises a set of non-visual indicators, such as an audio speaker, piezo electric element or other type of vibration feature, or other types of non-visual displays or indicators.

The set of logic circuits 12 may further comprise hardware, firmware, or software, for example, that may calculate and enforce or otherwise utilize an algorithm configured to translate health metrics from external devices, health data applications, or health services providers 18 into a health index value or score displayed in a visual or non-visual representation through the human interface 10. The device maybe comprised of one or more of a Read Only Memory (ROM), Programmable Read Only Memory (PROM), One Time Programmable Read Only Memory (OTP) to hold the private key, a specialized microcontroller to calculate the public address, a driver to calculate the display value and a Random Access Memory (RAM) to store the values and corresponding dates.

In another embodiment, human interface 10 may display text or symbols. In yet another embodiment, human interface 10 may transmit a light that comprises at least one of glowing, flashing, pulsing, vibrating, color-changing in intensity, color changing in hue, fading, and brightening.

In another embodiment, the apparatus may use blockchain technology to store historical health data through the use of an authenticated health metric data source's private key, e.g., every block's hash consists of previous block's hash, which is rehashed using the health metric data source's private key, and then in turn is used in the next block. For example, the apparatus may always read and display the health metric data, but none of the data in the existing blocks may be altered without invalidating the chain.

In yet another embodiment, human interface 10 is configured to display at least one of a video, animation, image, and other audiovisual and visual work.

In another embodiment, the apparatus may emit sounds, vibrations, pulses, or other non-visual auditory or kinetic indicators of a certain health index value, score or genealogical information of the wearer or change in the health index value, score or genealogical information. Much like visual indicators, these other auditory or kinetic indicators can be used to display a certain health index value or score, or any changes in the health index value or score.

Power source 20 may comprise a single use battery, a rechargeable battery, a photovoltaic cell, a solar battery, a heat battery, a motion generator, and a manual generator.

In another embodiment, the apparatus may comprise a set of drivers 14, logically configured to securely store and present information associated with an individual's health status (not shown), such as their genealogical information, health index value or score. The information may include an associated public key and a present value.

In another embodiment, the apparatus may comprise a set of medical monitoring devices 18, stored within the apparatus. In yet another embodiment, the apparatus may also include a set of connectors 16, wherein a connector of the set of connectors comprises a wireless and/or wired connector, operatively coupled to the set of logic circuits 12, and configured to logically connect the apparatus to a set of data sources 18, externally connected to the apparatus.

The set of data sources 18 may further be configured with at least one of an internal medical device or monitoring device (physically within apparatus 22), an Internet coupled external source of health metric data. The set of external health metric data may further comprise software, firmware, and/or hardware configured to store health metric information, such as such as a computer, smart telephone or tablet, or other hardware.

In one embodiment, a connector from the set of connectors 16 may also be used to recharge the apparatus via power source 20.

In another embodiment, a snapshot of a health index value or score, may be transferred to and stored on the apparatus 22, such as by capturing the then-current values derived from test results in a health data application or other health data repository (not shown in FIG. 1).

In another embodiment, in contrast to transferring the health index value or score to the apparatus, the health index value or score, not stored within the apparatus 22, may be represented through human interface 10 for a discrete period of time, before human interface 10 is refreshed to reflect the then current health index value or score.

In yet another embodiment, the apparatus may store and represent other types of health metrics not stored within apparatus 22, such as health data from a health services provider application, or from a genealogy information source.

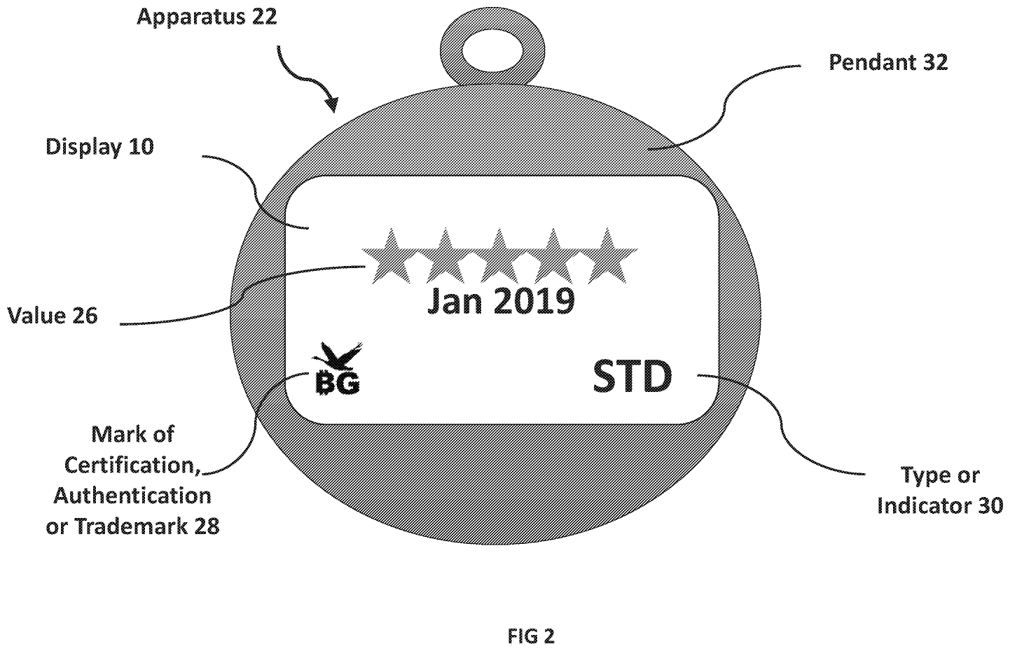

Referring to FIG. 2, a simplified diagram of one embodiment of the apparatus 22 is shown, in accordance with aspects of the present disclosure. In this configuration, the apparatus may be configured as a pendant 32 that may be prominently suspended round the neck of a wearer. Positioned on one face of pendant 32 may be a human interface, such as a display 10, configured to display text or symbols.

In another embodiment, the apparatus can be different types of jewelry or other wearable, such as a bracelet, necklace, watch, ring, pendant, key chain, arm band, etc.

In this example, the wearer's health status may be displayed, along with their health index score or genealogy indicator and the date or timestamp of when the health index score was last updated. In addition, a symbol 28 may be displayed, presenting a mark of authenticity of the health index value or score, or genealogy information, or a trademark of the organization that manages, controls, or stores the health index value or score.

A specialized microcontroller may be utilized to calculate the proper value of the display in accordance with the corresponding value of the wearer's health index value or score or genealogical information. An example would be that the display glows green for an individual in good health, yellow for an individual who may have some ongoing health conditions, or red for an individual in poor health. Furthermore, the intensity of the light may change within the range of each color, in accordance with sub-values of the wearer's health index value or certain types of medical conditions or health metrics. In another example, the display may change intensity and color in a periodic, dynamic, or rhythmic manner (pulse) to denote different kinds of health metrics, medical conditions, markers of physical fitness, the freshness of the data or other health metrics and can indicate a high level of physical fitness.

In another example, the color of the display may represent the type of healthy condition or health metrics (e.g., orange for resting heart rate, purple for blood pressure, green for the absence of any sexually transmitted diseases, or a symbol representing the type of health metric along with a value of the wearer's health index score). For example, the display may switch between different health conditions or health metrics stored within the apparatus and/or accessible by the apparatus. In addition, the display may mix and match various health conditions or health metrics stored within the apparatus and/or accessible by the apparatus, and thus, may result in a health index value or score that is a unique representation of the wearer's health status. The intensity, shine, and/or flashing may present the health index value or score or the status of different health conditions or health metrics (e.g., a decrease in blood pressure increases the intensity of illumination, and a blood pressure reading lower than 120/80 results in the light twinkling, shining, or flashing).

This approach allows the apparatus to be a physical representation of the wearer's health status or fitness or genealogical information, and allows the wearer to display that status in a conspicuous way, like fine jewelry, watches, or other traditional ways to display status.

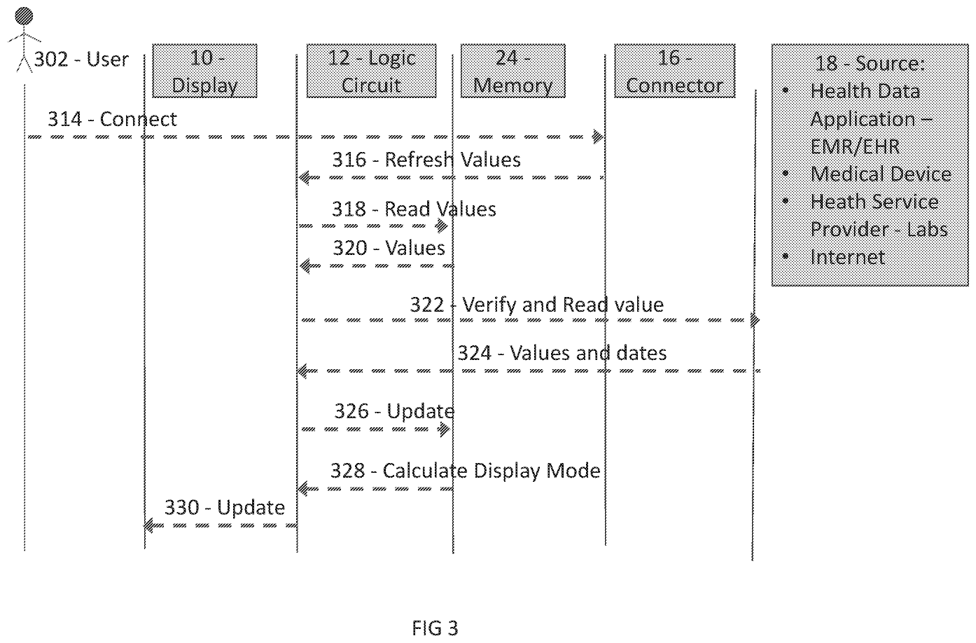

Referring to FIG. 3, a simplified process flow schematic that may be carried out by apparatus 22 of FIGS. 1 and 2 is shown, in accordance with aspects of the present disclosure. Initially, a user 302 connects the apparatus 22 (FIG. 1) to the source 18 (FIG. 1) in step 314 via connector 16 (FIG. 1), which maybe or include either a wired or a wireless connector, for example, coupled to one or more of the source 18 (FIG. 1) to read current values for the account, which in turn, connects to local memory 24 (FIG. 1).

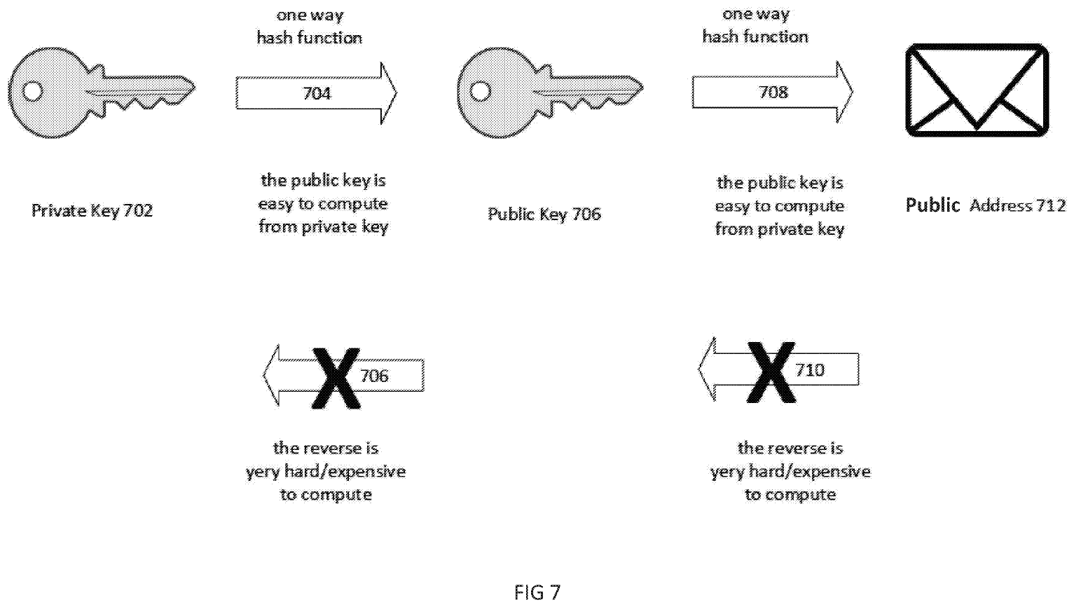

Health metric data is then transferred in step 316 from local memory 24 (FIG. 1) to logic circuit 12 (FIG. 1), which in turn, reads one or more of the keys 702 and 706 (FIG. 7) from local memory 24 (FIG. 1) associated with the previously retrieved values in step 318. The Public Key 706 (FIG. 7) and associated public address 712 (FIG. 7) are used to receive values or verify the latest health metrics to the account.

The keys and values are then transmitted in step 320 to logic circuit 12

The key or the address and the previously read value may be transmitted to connector 16 (FIG. 1), which in turn connects to the health metric data source 18 (FIG. 1), which may be located at one of a server or other device on the Internet, a medical device, a health data application, or a health service provider portal, for example, via step 322. Various healthcare data transfer protocols such as HL7 V2, V3 or FHIR may be employed for communication and transfer of the data. Logical circuit 12 (FIG. 1) may perform verifications to validate the values, dates and authenticity and accuracy of the data. Health Metric data may include, amongst others: cardiovascular conditions, blood pressure readings, glucose levels, Body Mass Index (BMI), weight, various enzyme levels (e.g., liver enzyme levels), various hormone levels (e.g., testosterone levels), various diseases (e.g., sexually transmitted diseases), corresponding statuses for these metrics, such as the contagiousness or remittance of a disease or condition, current or previous cancer diagnoses, DNA traits, such as an individual's predisposition to certain genetic diseases (e.g., Alzheimer's or Parkinson's disease), an individual's genealogical or and ancestral history, such as a person's relationship to certain races, tribes, ethnicities, countries, and associated percentages of each comprising the individual's genealogy or ancestry, environmental information, such as birth location or where the individual was raised and habitat information for each, an individual's age, fertility or menopausal status, family health history, relationships, activity information, and physical fitness information (e.g., V0₂ max, strength metrics, resting heart rate, etc.).

Verified values and dates are then transmitted back to display 10 (FIGS. 1 and 2) in step 324. Memory 24 (FIG. 1) is then updated by logic circuit 12 (FIG. 1) in step 326. Where the Memory 24 (FIG. 1) may be comprised of at one or more of a specialized Read Only Memory (ROM), Programmable Read Only Memory (PROM), One Time Programmable Read Only Memory (OTP) to hold the private key along and a Random Access Memory (RAM) to store the updated values and corresponding dates.

The display mode is then calculated in step 328, and the update is finally updated in step 330.

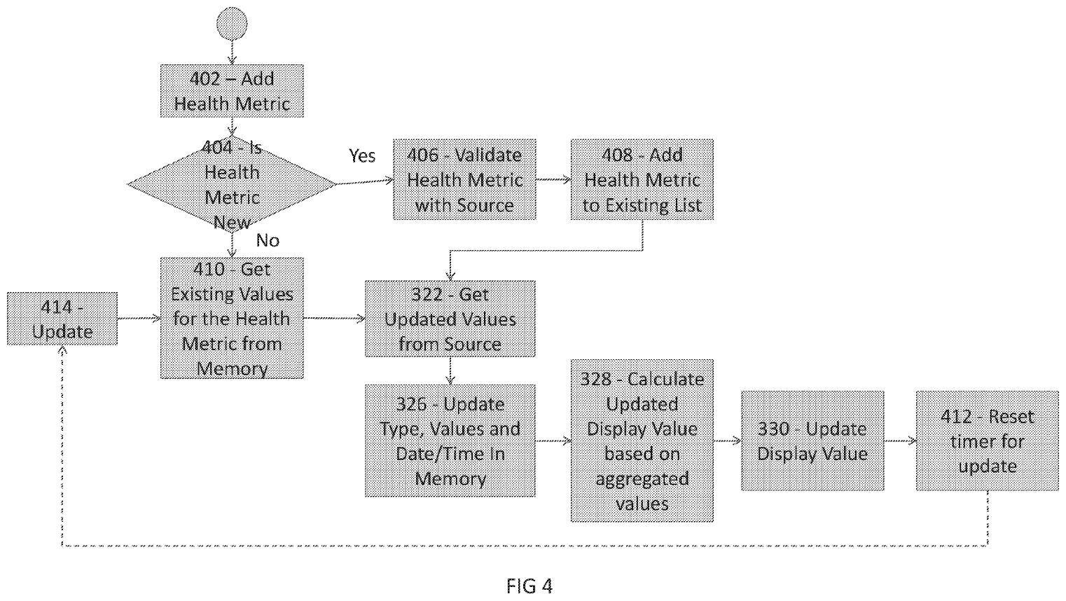

Referring now to FIG. 4, another representative flow diagram of the simplified process flow of FIG. 3 is shown, in which a health metric address is added to apparatus 22 (FIG. 1) and verified, in accordance with aspects of the present disclosure. A health metric address is a unique identifier of health data endpoints, for example, such as a health data source, a central server, or one or more apparatuses. Initially at step 402, health metric address is added to a memory 24 (FIG. 1) of apparatus 22 (FIGS. 1 and 2). Next at step 404, apparatus 22 (FIGS. 1 and 2) determines if the health metric address or source is new.

If the health metric address is new, the health metric address is validated with the source of the health metric address at step 406. The source may be, for example, accessible via the Internet and/or medical devices.

At 408, the health metric address is then added to an existing list in the memory on apparatus 22 (FIGS. 1 and 2). In an aspect, the existing list may be generated using blockchain technology (e.g., every block's hash in the existing list consists of a previous block's hash, which is rehashed using the private key of the health metric data source). Apparatus 22 (FIGS. 1 and 2) then gets updated values from the health metric address source as previously described.

If the health metric address or source is not new, the apparatus 22 (FIGS. 1 and 2) receives existing values for the health metric address from the memory (not shown) at step 410. As previously described, apparatus 22 (FIGS. 1 and 2) then gets updated values from the health metric address source at step 322. Apparatus 22 (FIGS. 1 and 2) then updates the type, values and date/time in memory 326.

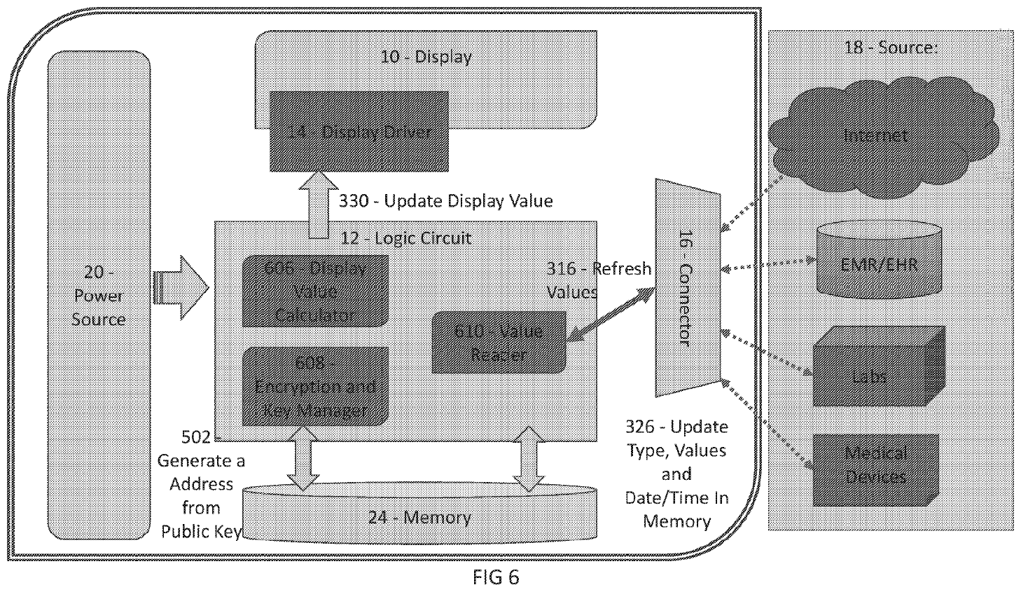

The display value, which may be a health index value or score, is then calculated at 328 based on aggregate values through specialized Logic Circuit 12 (FIG. 1) comprised of the Memory 24 (FIG. 1) and Display Value Calculator 606 (FIG. 6).

The updated value is then displayed in human interface 10 (FIGS. 1 and 2) at step 330. The updated value may be displayed by any or all of the indicators described above, e.g., light, vibration, sound, etc.

Finally, an update timer in apparatus 22 (FIGS. 1 and 2) is reset at step 412.

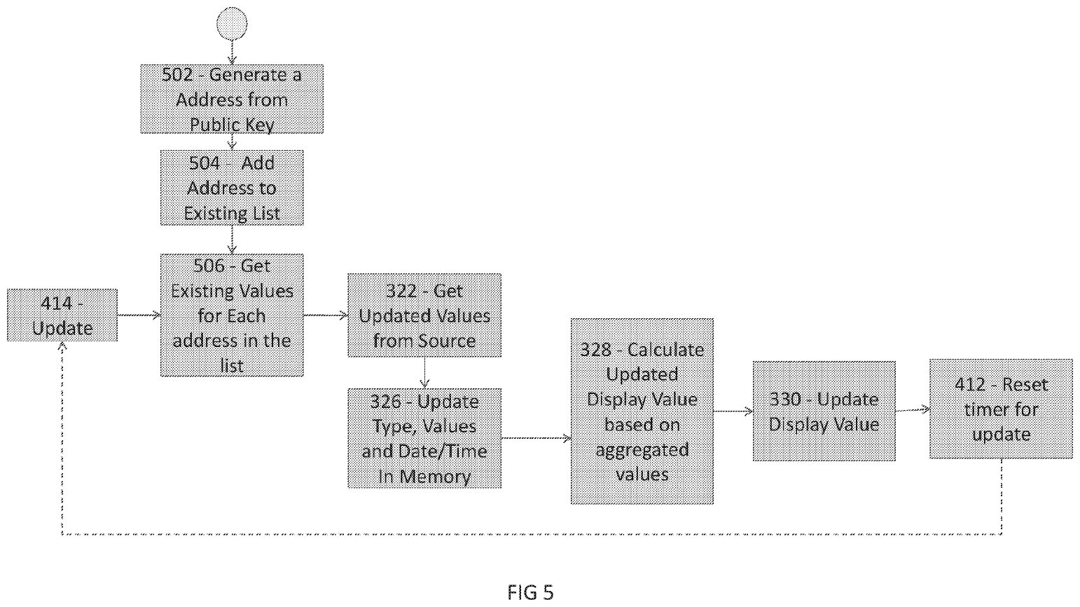

Referring now to FIG. 5, a process for updating a set of values of apparatus 22 (FIGS. 1 and 2) from a health metric source with an address generated from a public key is shown, in accordance with aspects of the present disclosure.

Initially, a health metric source address is generated from a public key (if needed) at step 502. Next, the health metric source address is added to an existing list in a memory 24 (FIG. 1) of apparatus 22 (FIG. 1) at step 504. In an aspect, the existing list may be generated using blockchain technology (e.g., every block's hash in the existing list consists of a previous block's hash, which is rehashed using the private key of the health metric data source).

Next, at step 506, existing values for each health metric address in existing list are retrieved.

Next, values from each health metric source may be updated in step 322.

Next health metric type, values, and the date/time are updated in memory 24 (FIG. 1) of apparatus 22 (FIGS. 1 and 2).

Next, an updated display value is calculated based on aggregated values 328.

Next the display value in display 10 (FIGS. 1 and 2) is updated at step 330.

Next, the time is reset at step 412, for the next update 414.

Referring now to FIG. 6, a simplified logical diagram of the apparatus 22 (FIGS. 1 and 2) is shown in accordance with aspects of the present disclosure.

In general, as shown in FIG. 1, apparatus 22 is configured with a logic circuit 12 logically coupled to display driver 14, power source 20, memory 24, a connector 16. Display driver 14 is further logically coupled to display 10.

As shown in FIG. 6, encryption and key manager 608, coupled to memory 24 (FIG. 1), is configured to generate a digital asset address from a public digital asset key 502. Value reader 610 is configured to update in memory 24 (FIG. 1) the health metric type, values, and date/time 326.

The health metric value reader 610 is further logically coupled to connector 16 (FIG. 1) and is configured to refresh health metric values. Connector 16 (FIG. 1) is further logically coupled to the health metric data source 18 (FIG. 1), which may comprise one or more of a server of other device accessible via the Internet, or an otherwise coupled node or health data application or medical devices, for example.

FIG. 7 shows a representative diagram of a one-way hash functions 704, 708 that may be used with health metrics. In general, a private key 702 may be generated that may be further used to generate a single public key 706 via a one-way hash function which, in turn, may be used to generate a set of health metric addresses. In general, it is computationally difficult to reverse the calculation as can be seen in step 710 or step 706.

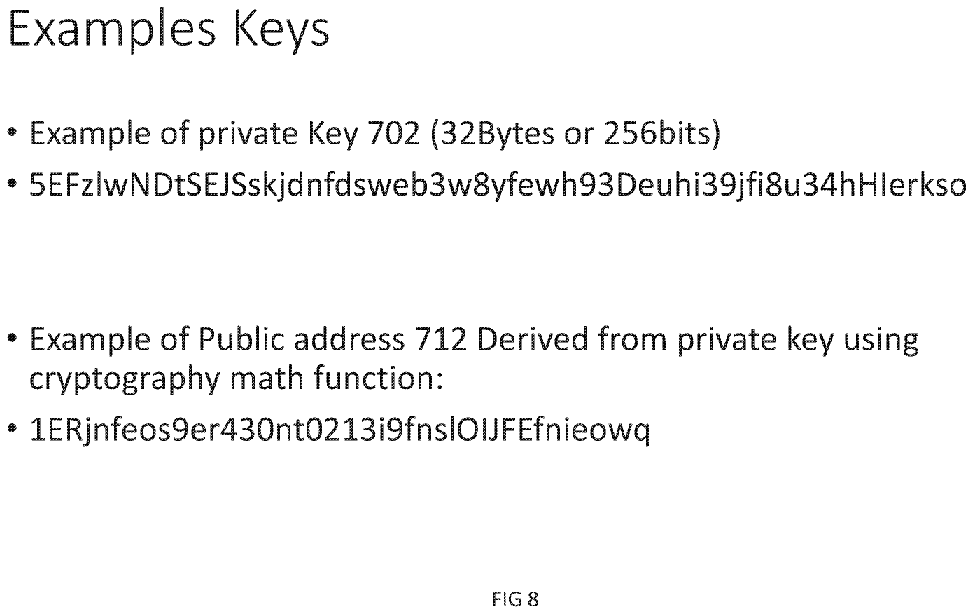

Referring now to FIG. 8, an example of a private health metric key and health metric public address usable in accordance with various aspects of the present disclosure are shown.

The private and public keys are mainly used for encryption, verification and authentication of data as well as the address of the end points for the transmission of data.

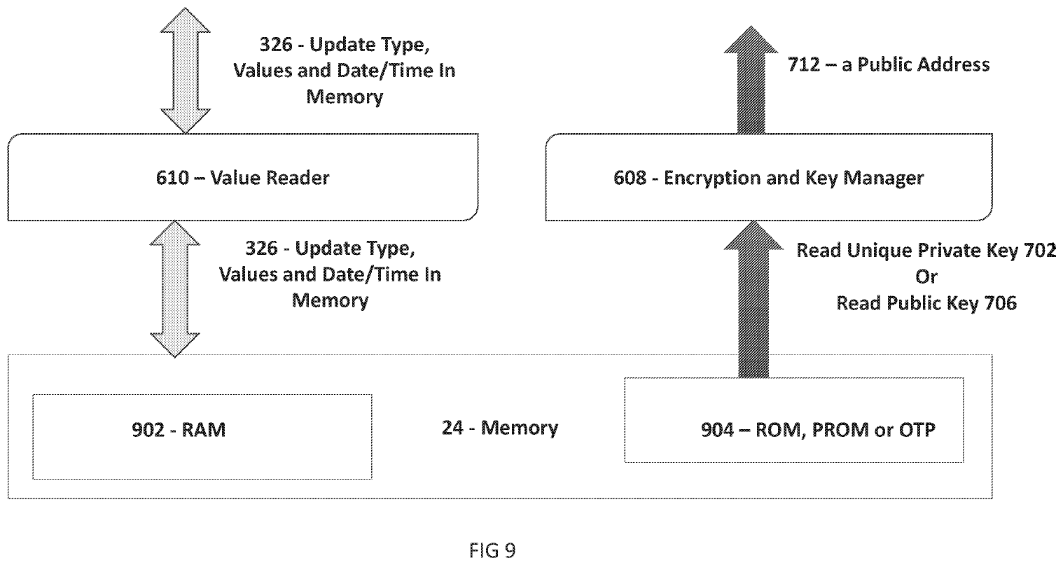

FIG. 9 shows a representative diagram illustrating how a specialized memory, such as at least one of a Read Only Memory (ROM), Programmable Read Only Memory (PROM), One Time Programmable Read Only Memory (OTP), or Random Access Memory (RAM) 904, can be utilized to store and/or otherwise use a Private Key 702 and Public Key 706. Encryption and key manager 608 can access these values directly or indirectly to derive the Public address 712.

In one embodiment, encryption and key manager 608 can be so tightly coupled with the ROM 904 that the private key 702 become inaccessible externally. In such embodiment, any values transferred to the public address 712 corresponding to the inaccessible private key 702 will not be alterable once saved in the apparatus 22. This feature will allow the apparatus 22 to forever hold on to the health metric data transferred into it, for example.

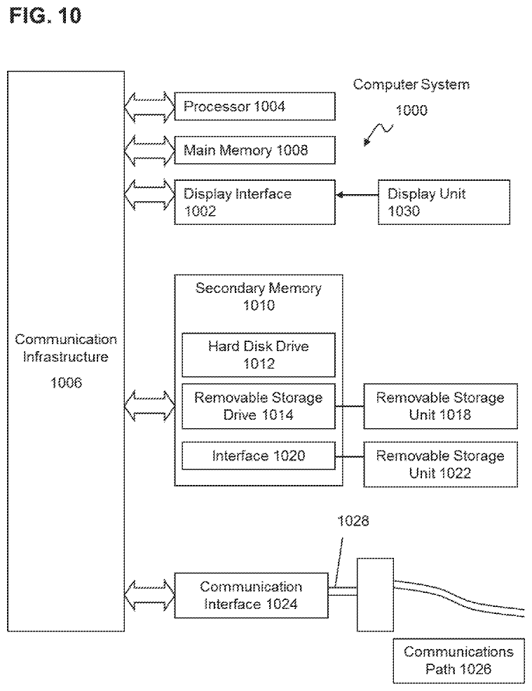

Aspects of the present disclosure may be implemented using hardware, software, or a combination thereof and may be implemented in one or more computer systems or other processing systems. In an aspect of the present disclosure, features are directed toward one or more computer systems capable of carrying out the functionality described herein. An example of such a computer system 1000 is shown in FIG. 10.

Computer system 1000 includes one or more processors, such as processor 1004. The processor 1004 is coupled to a communication infrastructure 1006 (e.g., a communications bus, cross-over bar, or network). Various software aspects are described in terms of this example computer system. After reading this description, it will become apparent to a person skilled in the relevant art(s) how to implement aspects hereof using other computer systems and/or architectures.

Computer system 1000 may include a display interface 1002 that forwards graphics, text, and other data from the communication infrastructure 1006 (or from a frame buffer not shown) for display on a display unit 1030. Computer system 1000 may include a main memory 1008, preferably random access memory (RAM), and may also include a secondary memory 1010. The secondary memory 1010 may include, for example, a hard disk drive 1012 and/or a removable storage drive 1014, e.g., an optical disk drive. The removable storage drive 1014 may read from and/or write to a removable storage unit 1018 in a well-known manner. As will be appreciated, the removable storage unit 1018 may include a computer readable storage medium having stored therein computer software and/or data.

Alternative aspects of the present disclosure may include secondary memory 1010 and may include other similar devices for allowing computer programs or other instructions to be loaded into computer system 1000. Such devices may include, for example, a removable storage unit 1022 and an interface 1020. Examples of such may include a program cartridge and cartridge interface (such as that found in video game devices), a removable memory chip (such as an erasable programmable read only memory (EPROM), or programmable read only memory (PROM)) and associated socket, and other removable storage units 1022 and interfaces 1020, which allow software and data to be transferred from the removable storage unit 1022 to computer system 1000.

Computer system 1000 may also include a communications interface 1024. Communications interface 1024 may allow software and data to be transferred among computer system 1000 and external devices. Examples of communications interface 1024 may include a modem, a network interface (such as an Ethernet card), a communications port, a Personal Computer Memory Card International Association (PCMCIA) slot and card, etc. Software and data transferred via communications interface 1024 may be in the form of signals 1028, which may be electronic, electromagnetic, optical or other signals capable of being received by communications interface 1024. These signals 1028 may be provided to communications interface 1024 via a communications path (e.g., channel) 1026. This path 1026 may carry signals 1028 and may be implemented using wire or cable, fiber optics, a telephone line, a cellular link, a radio frequency (RF) link and/or other communications channels. As used herein, the terms “computer program medium” and “computer usable medium” refer generally to media such as a removable storage drive 1014, a hard disk installed in hard disk drive 1012, and/or signals 1028. These computer program products may provide software to the computer system 1000. Aspects of the present disclosure are directed to such computer program products.

Computer programs (also referred to as computer control logic) may be stored in main memory 1008 and/or secondary memory 1010. Computer programs may also be received via communications interface 1024. Such computer programs, when executed, may enable the computer system 1000 to perform the features in accordance with aspects of the present disclosure, as discussed herein. In particular, the computer programs, when executed, may enable the processor 1004 to perform the features in accordance with aspects of the present disclosure. Accordingly, such computer programs may represent controllers of the computer system 1000.

Where aspects of the present disclosure may be implemented using software, the software may be stored in a computer program product and loaded into computer system 1000 using removable storage drive 1014, hard disk drive 1012, or interface 1020. The control logic (software), when executed by the processor 1004, may cause the processor 1004 to perform the functions described herein. In another aspect of the present disclosure, the system may be implemented primarily in hardware using, for example, hardware components, such as application specific integrated circuits (ASICs). Implementation of the hardware state machine so as to perform the functions described herein will be apparent to persons skilled in the relevant art(s). In yet another variation, aspects of the present disclosure may be implemented using a combination of both hardware and software.

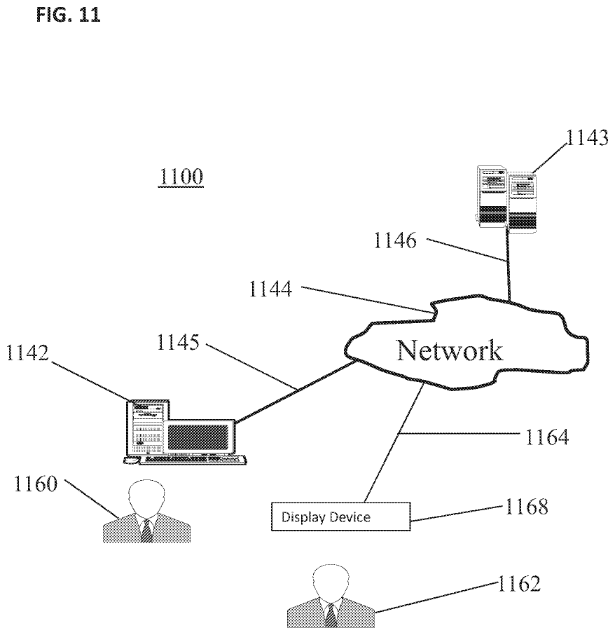

FIG. 11 shows a communication system 1100 usable with various features in accordance with aspects of the present disclosure. The communication system 1100 includes one or more accessors 1160, 1162 (also referred to interchangeably herein as one or more “users”) and one or more terminals 1142, 1168, such as the display device 22 shown and described with respect to FIGS. 1-9. In one aspect of the present disclosure, data for use is, for example, input and/or accessed by accessors 1160, 1162 via terminals 1142, 1168, such as device 22 of FIGS. 1-9, personal computers (PCs), minicomputers, mainframe computers, microcomputers, telephonic devices, or wireless devices, personal digital assistants (“PDAs”) or a hand-held wireless devices (e.g., wireless telephones) coupled to a server 1143, such as a PC, minicomputer, mainframe computer, microcomputer, or other device having a processor and a repository for data and/or connection to a repository for data, via, for example, a network 1144, such as the Internet or an intranet, and/or a wireless network, and couplings 1145, 1146, 1164. The couplings 1145, 1146, 1164 include, for example, wired, wireless, or fiberoptic links. In another aspect of the present disclosure, the method and system of the present disclosure may include one or more features that operate in a stand-alone environment, such as on a single terminal.

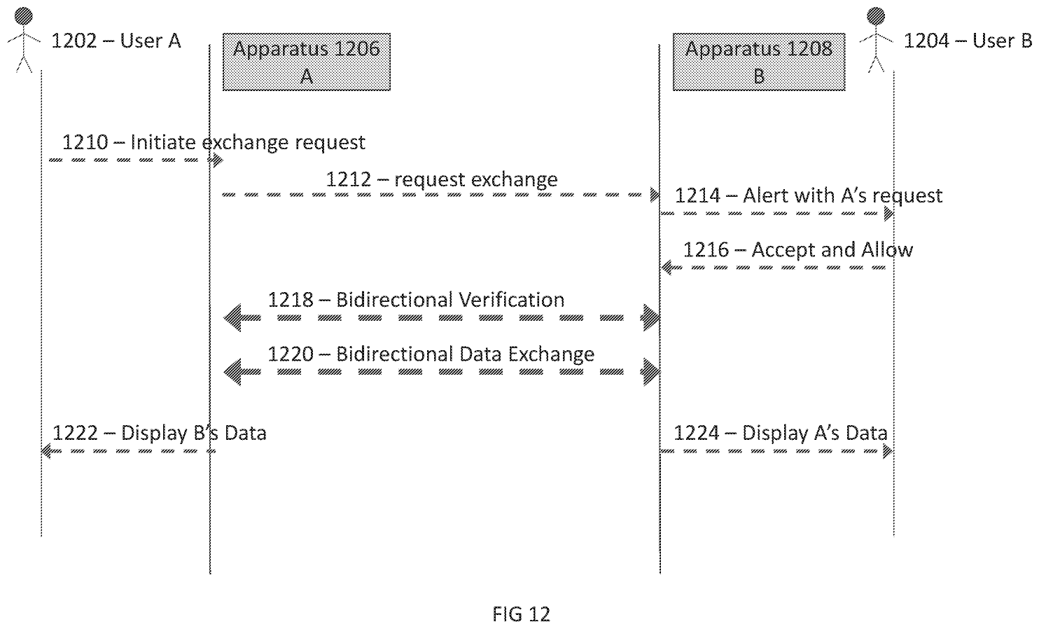

FIG. 12 is a diagram of how one apparatus interacts with one or more apparatuses to exchange privately exchange health status information between individuals. A user 1202 initiates a request 12010 on their apparatus 1206 to exchange health status information or a health index value or score with another user's apparatus 1208. The requesting apparatus 1206 then sends an exchange request 1212 to the recipient's apparatus 1208. This request may happen locally though technology, such as Bluetooth, or over the Internet. The request 1212 appears as an alert 1214 on the recipient's apparatus 1208. The recipient can accept or deny the request 1214.

When the recipient accepts a request 1216, there is a bi-directional verification procedure 1218 that occurs between the initiating apparatus 1206 and recipient apparatus 1208 and which authenticates the other apparatus and any associated addresses for the purpose of securely exchanging health data. This verification procedure, for example, may be comprised of Bluetooth pairing of a four to six digit numbers between each apparatus. Next, after the verification step 1218 is completed, health data or health index scores or values are exchanged 1220 between the requesting apparatus 1206 and the recipient apparatus 1208. After the health data is exchanged, each apparatus displays the health data or health index scores of the other apparatus. For example, the original requesting apparatus 1206 displays the health data 1222 of the original recipient apparatus 1208, and the original recipient apparatus 1208 displays the health data 1224 of the original requesting apparatus 1206.

Example of such use case may be exchanging subset of health data with another person to ensure they are clear from contagious diseases before interacting closely with them or ensure of DNA or blood compatibility with a prospect sexual or life partner.

In another aspect of the present disclosure, this exchange of health data may happen between a group of apparatuses. In yet another aspect of the present disclosure, apparatus owners may also select what data, subset of data, or individual health metrics they would like to exchange. In a further aspect of the present disclosure, an apparatus owner may be required to reciprocate the permission to disclose the same health data as requesting apparatus owner before an exchange can occur. For example, if an apparatus owner requests the health index score of another apparatus owner, the requesting apparatus owner must also share their health index score with the party from whom they have requested the health index score.

An example of such use case is to share the subset of health data with sports team or employers for gaming or verification purposes.

In all cases above, the information about the type of the device (immutable history or not) maybe communicated as well with individuals or group for verification purposes.

While the foregoing disclosure discusses example aspects and/or features, it should be noted that various changes and modifications could be made herein without departing from the scope of the described aspects and/or features as defined by the appended claims. Furthermore, although elements of the described aspects and/or features may be described or claimed in the singular, the plural is contemplated unless limitation to the singular is explicitly stated. Additionally, all or a portion of any aspect and/or embodiment may be utilized with all or a portion of any other aspect and/or feature, unless stated otherwise.

1. An apparatus for presenting health status of an individual, the apparatus comprising:
a first health metric data receiver configured to receive one or more current values associated with at least one first health metric endpoint or source;
a first microcontroller configured to perform a calculation using the one or more current values to determine a first value of the at least one first health metric endpoint or source;
a first human interface configured to select a first display that represents the value of the at least one first health metric endpoint or source from a plurality of predefined displays, where each of the plurality of predefined displays represents a different value of health metrics, and present the first display;
an attaching part to attach the apparatus to a wearer;
an input device for authenticating the wearer; and
a set of connectors for connecting with at least one other apparatus, the at least one other apparatus comprising:
a second health metric data receiver configured to receive one or more current values associated with at least one second health metric endpoint or source;
a second microcontroller configured to perform a second calculation using the one or more current values to determine a second value of the at least one second health metric endpoint or source;
a second human interface configured to select a second display that represents the second value of the at least one second health metric endpoint or source from the plurality of predefined displays, where each of the plurality of predefined displays represents a different value of health metrics, and present the second display;
a second attaching part to attach the second apparatus to a different wearer; and
a second input device for authenticating the different wearer.

2. The apparatus of claim 1, wherein the at least one first health metric endpoint or source is one or more of a healthcare provider, a central server, a wearable device, a medical device, a health data application, or a health services application, and
wherein the at least one first health metric endpoint or source includes a private key that is used to generate a public key and a public address for the at least one first health metric endpoint or source.

3. The apparatus of claim 1, wherein the first microcontroller is further configured to:
determine a health metric type for the at least one first health metric endpoint or source; and
aggregate the one or more current values based at least on the health metric type to determine the value of the at least one first health metric endpoint or source.

4. The apparatus of claim 1, wherein the first human interface is configured to select the first display from the plurality of predefined displays when the value of the at least one first health metric endpoint or source is within a range of values associated with the first display.

5. The apparatus of claim 1, wherein the first microcontroller is further configured to perform a revised calculation of the one or more current values after a predetermined period of time;
the first human interface is further configured to select the first display using the revised calculation of the value; and
wherein the apparatus further includes a memory storage unit configured to store historical values of the at least one first health metric endpoint or source using an authenticated private key associated with the at least one first health metric endpoint or source to create a hash of the historical values.

6. The apparatus of claim 1, wherein the first display includes one or more of glowing, flashing, pulsing, changing color, vibrating, color changing in intensity, fading in, fading out, changing brightness, changing hues, electronic ink, electronic paper, haptic feedback, or an aural representation.

7. The apparatus of claim 1, wherein the value of the at least one first health metric is one or more of a disease test result, health condition test result, DNA test result, blood type test result, blood pressure reading, V0₂ max reading, blood sugar index reading, body mass index reading, or resting heart rate reading.

8. The apparatus of claim 1, further comprising:
a memory storage unit configured to securely store the at least one first health metric endpoint or source.

9. The apparatus of claim 1, wherein the at least one first health metric endpoint or source is stored in a health data application or health services application, medical device, or an account remote from the apparatus.

10. The apparatus of claim 1, wherein the first health metric data receiver is further configured to:
verify a source of a health metric address associated with the at least one first health metric endpoint or source using a public key associated with the health metric address; and
update the one or more current values associated with the at least one first health metric endpoint or source when the source of the health metric address is verified.

11. The apparatus of claim 1, further comprising:
a memory storage unit configured to securely store an externally inaccessible private key in the apparatus for the at least one first health metric endpoint or source; and
an encryption and key manager configured to associate the externally inaccessible private key with a public key or public address for the at least one first health metric endpoint or source.

12-24. (canceled)
